(19) United States
(12) Patent Application Publication
(10) Pub. No.: US 2009/0319034 A1
(43) Pub. Date: Dec. 24, 2009

(54) METHOD OF DENSIFYING ePTFE TUBE

(75) Inventor: Krzysztof Sowinski, Wallington, NJ (US)

Correspondence Address: KRZYSZTOF SOWINSKI;BOSTON SCIENTIFIC SCIMED, INC.,, ONE SCIMED PLACE, MAPLE GROVE, MN, 55311-1566, US

(73) Assignee: BOSTON SCIENTIFIC SCIMED, INC, Maple Grove, MN (US)

(21) Appl. No.: 12/142,144

(22) Filed: Jun. 19, 2008

(51) Int. Cl. A61F 2/82; B29C 59/04

(52) U.S. Cl. 623/1.46; 264/175

(57) ABSTRACT

The present invention relates to a prosthetic structure, including a densified layer of expanded polytetrafluoroethylene (ePTFE) and a method for manufacturing the same. The invention includes the steps of providing a layer of ePTFE, desirably a tubular layer; applying the layer of ePTFE to a mandrel; mechanically compressing the layer of ePTFE on the mandrel; and removing the compressed ePTFE; where the compressed ePTFE is denser than uncompressed ePTFE. The compressed ePTFE has a water entry pressure (WEP) value of at least 15 psi, and desirably a WEP of at least about 20 psi.

### FIELD OF THE INVENTION

The present invention relates to densified expanded polytetrafluoroethylene (ePTFE) products and methods of their manufacture. The present invention further relates to medical devices such as grafts, endoprosthesis or intraluminal devices, which include as a component densified ePTFE material. Densified ePTFE has a low permeability, allowing for an improved use as an implantable prosthetic. Densified ePTFE as described herein may be used as a base layer for stent coverings which require low permeability.

### BACKGROUND OF THE INVENTION

It is well known to use extruded tubes of polytetrafluoroethylene (PTFE) as implantable intraluminal prostheses, particularly as vascular grafts. PTFE is particularly suitable as an implantable prosthesis as it exhibits superior biocompatibility. PTFE prosthetics may be any shape or design desired, and includes tubes, patches, tapes, sheets and the like. Further, PTFE tubes may be bifurcated into y-shaped tubes. PTFE tubes may be used as vascular grafts in the replacement or repair of a body vessel as PTFE exhibits low thrombogenicity. Such prosthetic tubes may be used to replace, reinforce, or bypass a diseased or injured body lumen.

Often, prosthetics are made of expanded PTFE, which exhibits superior biological effectiveness. One conventional method of manufacturing “expanded” PTFE (ePTFE) is described in U.S. Pat. No. 3,953,566 to Gore. In the methods described therein, a PTFE paste is formed by combining a PTFE resin and a lubricant. The PTFE paste may be extruded. After the lubricant is removed from the extruded paste, the PTFE article is stretched to create a porous, high strength PTFE article. The expanded PTFE layer is characterized by a porous, open microstructure, which has interspaced nodes of PTFE interconnected by fibrils.

Structures formed of ePTFE exhibit certain beneficial properties as compared with textile prostheses. The space between the PTFE nodes, which are spanned by fibrils is defined as the internodal distance (IND). The expansion of PTFE increases the volume of the PTFE layer by increasing the porosity, which results in a decrease in the density and increase in the internodal distance between adjacent nodes in the microstructure. The node and fibril structure defines pores in the structure that facilitate a desired degree of tissue ingrowth while remaining substantially fluid-tight.

In particular, tubes of ePTFE may be formed to be exceptionally thin, and yet exhibit the requisite strength necessary to serve in the repair or replacement of a body lumen. The thinness of the ePTFE tube facilitates ease of implantation and deployment with minimal adverse impact on the body. These tubes have a microporous structure which allows natural tissue ingrowth and cell endothelization once implanted in the vascular system. This node and fibril structure helps contribute to long term healing and patency of the graft.

Typically, porous expanded PTFE is desirable to promote tissue growth. However, the pores in ePTFE may undesirably weaken the structure and make the ePTFE susceptible to leakage through the walls of the prosthetic. The tendency of the structure to leak through the pores is measured by examining the water entry pressure required to leak (commonly referred to as a “WEP test”). Such structures may in turn create a phenomenon known as ultra-filtration which can ultimately lead to an increase in the abdominal aortic aneurysm diameter. As such, there is a need in the art for a strong ePTFE tube with very low or zero permeability and a water entry pressure of at least 20 psi.

### SUMMARY OF THE INVENTION

In one embodiment, there is contemplated a method for manufacturing compressed ePTFE, including the steps of providing a layer of ePTFE, desirably a tubular layer; applying the layer of ePTFE to a mandrel; mechanically compressing the layer of ePTFE on the mandrel; and removing the compressed ePTFE; where the compressed ePTFE is denser than uncompressed ePTFE. The compressed ePTFE has a water entry pressure (WEP) value of at least 15 psi, and desirably a WEP of at least about 20 psi. The density of the resultant ePTFE layer is about 0.4 to about 2 grams/cc; specifically, from about 1.0 to about 2.0 grams/cc.

In another embodiment, there is provided an ePTFE tube, including at least one layer of compressed ePTFE, wherein the ePTFE has a WEP value of at least about 20 psi and a density of from about 1.0-2.0 grams/cc.

In another embodiment, there is provided a prosthetic structure, including at least one layer of ePTFE, wherein the ePTFE includes a WEP value of at least about 20 psi and a density of about 1.0-2.0 grams/cc.

In yet another embodiment, there is provided an apparatus for conveying fluid, which includes an ePTFE tube, the ePTFE tube including at least one layer of compressed ePTFE, wherein the ePTFE has a WEP value of at least about 20 psi and a density of from about 1.0-2.0 grams/cc.

In another embodiment of the invention, there is provided a means for conveying fluid, which includes an ePTFE tube, said ePTFE tube including at least one layer of compressed ePTFE.

### BRIEF DESCRIPTION OF THE DRAWINGS

FIG. 1 is a cross-sectional drawing of the mechanical compressor described herein.

FIG. 2 is a photograph of the mechanical compressor of the present invention.

FIG. 3 is a schematic description of one embodiment of the process of the present invention.

### DETAILED DESCRIPTION OF THE INVENTION

As used herein, the term “PTFE” refers to polytetrafluoroethylene, and can refer to any structure made at least partially of polytetrafluroethylene. The term “ePTFE” refers to expanded polytetrafluoroethylene, and can refer to any structure made at least partially of expanded polytetrafluoroethylene. Both PTFE and ePTFE may be manufactured by any method desired. As also used herein, the term “densified” refers to a structure that has been altered in some fashion so as to increase its density. The term “densification” refers to the process for increasing density in the structure by any means desired. Any increase in density desired may be incorporated. Preferably, a structure that has been “densified” as used herein has a density that is at least slightly denser than the original structure prior to the “densification.” The increase in density is intended to achieve a material which has less tendency to permit water permeation under pressure. A lower WEP value means that the level of pressure required for water to leak through the structure is lower. The densified materials of the present invention desirably have a WEP value of at least 15 psi at room temperature, and most preferably at least about 20 psi. This is a substantial increase in WEP value as compared to known uncompressed ePTFE tubular structures, which generally have a WEP value of about 2 to about 5 psi.

The present invention includes an ePTFE structure that has an increased density. In addition to testing the WEP value of the products, density may be measured by any means desired, including use of a gas pycnometer or other density measurement tool. A structure of PTFE is formed and expanded to form ePTFE. Expansion may be achieved by any desired method. The ePTFE structure may be of any shape desired, and may in one embodiment be tubular in shape. The structure may optionally be in the shape of a patch, a sheet, or a tape, or combinations thereof. The structure may be a multi-tubular shape, such as a split tube, which is commonly used for repairing abdominal aortic aneurysms (AAA) or thoracic aortic aneurysms (TAA), or it may be a multi-layered design, such as a tube-in-tube design. The tubular structure may be of any shape desired, and may include circular, oval, tapered, flared, or combinations thereof. Any ePTFE structure may be used as a prosthetic device, including tubes, patches, sheets, tapes, or combinations thereof, and may be used to convey fluids, such as blood or other bodily fluid.

Once the ePTFE structure is formed, the structure may be densified by any means desired. In one embodiment, the structure is densified via mechanical compression. Any mechanical compressor may be used to impart a force, or combination of forces, on the ePTFE structure. The force should be great enough to increase the density of the structure while not being so excessive as to rupture or tear the structure.

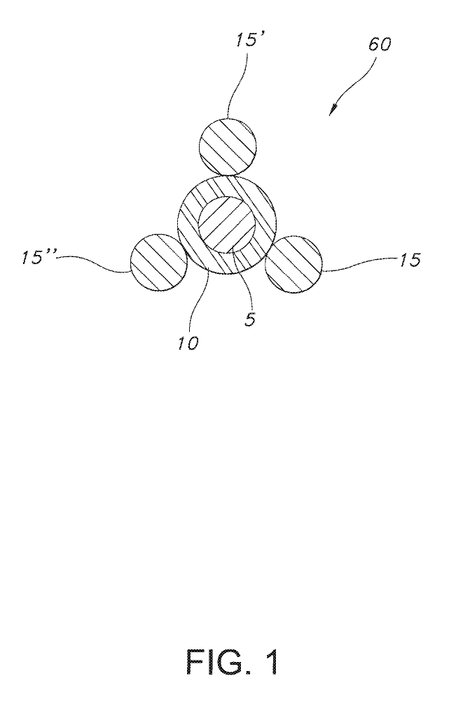

The forces may be circumferential, tangential, or radial. In one embodiment, depicted in FIG. 1, the mechanical compressor 60 incorporates multiple pressure rollers 15, 15′, 15″, which impart forces on an ePTFE structure 10. Any number of pressure rollers 15 may be incorporated, depending on the size of the compressor and the amount of pressure desired to be exerted. The pressure rollers 15 are preferably located on the outside of the ePTFE structure 10, but may be located on the inside surface of the ePTFE structure 10 if desired. In the embodiment shown in FIG. 1, the ePTFE structure 10 is tubular in shape, and is located on the outer surface of a rod-shaped mandrel 5, so that the outer surface of the mandrel 5 is in contact with the inside surface of the ePTFE structure 10. The pressure rollers 15 are located on the outer surface of ePTFE structure 10. In this embodiment, the pressure rollers 15 exert pressure on the ePTFE structure 10, pushing the outer surface of the ePTFE structure 10 towards the inside, against the mandrel 5. In one embodiment, the pressure rollers 15 are rotatable around the outer surface of the ePTFE structure 10, such that a pressure may be exerted circumferentially on the ePTFE structure 10.

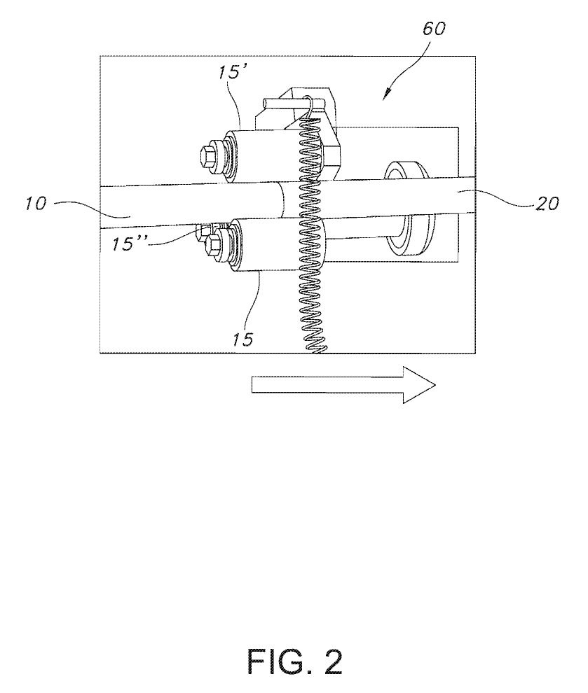

As the ePTFE structure 10 is compressed via exertion of pressure by the pressure rollers 15, the ePTFE structure 10 moves along the axial direction, allowing for compression along the length of the ePTFE structure 10. As can be seen more clearly in FIG. 2, the ePTFE structure 10 moves along the axial direction through the mechanical compressor 60. Prior to entering the mechanical compressor 60, the ePTFE structure 10 is not compressed, and demonstrates the traditional node and fibril structure associated with ePTFE in general. In the mechanical compressor 60, the ePTFE structure 10 is compressed via a plurality of pressure rollers 15, 15′. Any number of pressure rollers 15 may be used. As the ePTFE structure 10 moves along the mechanical compressor 60, the pressure rollers 15 may move circumferentially about the ePTFE structure 10, to compress the ePTFE structure 10 around the circumference. After compression, a compressed ePTFE structure 20 exits the mechanical compressor 60. While the embodiment shown in FIGS. 1 and 2 depicts a tubular ePTFE structure 10, it is contemplated that any shape and style of ePTFE structure may be incorporated, including a flat or sheet-like structure, or other desired shape. The ePTFE structure 10 may remain in the compressor 60 for any desired length of time, and may vary from any length of time from 10 seconds to 5 minutes.

The mechanical compressor 60 may be heated to a higher temperature to aid in the compression of the ePTFE structure 10. In particular, the compressor 60 may be heated to a temperature between about 100° F. and about 300° F., and specifically between about 120° F. and about 160° F.

In one embodiment, the ePTFE structure is first applied to the surface of a mandrel, followed by densification, such as by mechanical compression described above. In another embodiment, the ePTFE structure may be tubular in shape, and may be applied to the outside of a rod-shaped mandrel so that the inner surface of the ePTFE structure is in contact with the outer surface of the mandrel. Once applied to the outer surface of a tubular mandrel, the ePTFE structure may be subjected to pressure from outside surface of the ePTFE structure via the mechanical compressor. Alternatively, the compression may be from the inside of the structure, pushing outward. In this alternative embodiment, the outer surface of the structure is in contact with the inner surface of the mandrel.

Densifying the structure via subjecting the ePTFE structure to mechanical compression results in a physical change to the ePTFE structure. As described above, after extrusion, ePTFE has a porous structure, identified by individual nodes of PTFE that are interconnected via web-like fibrils of PTFE. Such porous structure may undesirably allow fluid to pass through the structure, particularly under pressure. For example, when the densified material of the present invention is used as a vascular graft, the blood pressure and constant forces placed upon the ePTFE material can cause slow permeation of the blood through less dense areas over time. This is particularly the case when stent structures are placed between layers of the polymer material and laminated together, since the tent-like structure formed the ePTFE around the stent are generally single layers rather than laminates. Other causes for potential seeping of fluid through the material under pressure can result from areas which are not fully laminated together of are not laminated at all. For example, in cases where the mandrel has holes or dimples, such as for the purpose of pulling a vacuum on the material against the mandrel, the portions of the material which cover the holes have a tendency to get sucked into the hole. Subsequently, the lamination of the layers in that area will be less dense than portions which are directly against the mandrel. Uniformity of density in prosthetic structures such as vascular and endovascular grafts and stent-grafts is important, particularly where arterial pressures are concerned.

After mechanical compression, as described herein, the nodes and fibrils of the structure become compressed together, forming a structure that is denser than prior to compression. In fact, in some embodiments, the mechanical compression may compress the material in such a way that the nodes and fibrils are minimized or can no longer be identifiable. In one embodiment, after mechanical compression, the ePTFE structure no longer demonstrates the porous nodular/fibril structure commonly seen in ePTFE structures. In this embodiment, the ePTFE structure compressed via the process of the present invention has no identifiable nodes or fibrils. After compression, the ePTFE structure is highly densified and exhibits a high WEP value.

In some embodiments, the compressed ePTFE structure may exhibit no nodes and fibrils. Other embodiments may still exhibit a slightly porous structure, having identifiable nodes and fibrils. Any degree of compression and densification desired may be incorporated, and may be achieved by varying the amount of pressure exerted on the ePTFE structure. Some degree of porosity may be desired, which may be achieved by using controlled compression and pressure levels. In some embodiments, a highly porous ePTFE structure may remain after compression by exerting a small amount of pressure, while in other embodiments a low-porous or non-porous ePTFE structure may remain after compression by exerting a high amount of pressure.

Compression of the ePTFE structure results in a denser structure than was present prior to compression. Any degree of density increase may be achieved. The compressed structure may be any density from about 1 times as dense as uncompressed ePTFE to more than 40 times as dense as uncompressed ePTFE. Preferably, the compressed ePTFE structure has a level of density from about 0.5 to about 2.0 grams/cc, and most desirably about 1.0 to about 1.5 grams/cc. Desirably, the compressed ePTFE structure is about 3 to about 10 times as dense as a non-compressed structure. Like the varying degree of porosity as described above, the density may be varied by changing the amount of pressure exerted during compression. The compressed ePTFE structure preferably experiences an increase in its WEP value, indicating a denser, less porous structure. In a preferred embodiment, the structure has a WEP value of at least about 15 psi, and more particularly has a WEP value of at least about 20 psi. If desired, the compression may take place along the entire ePTFE structure, or may be at select locations on the structure. This may be achieved by placing the selected location(s) of the ePTFE structure on the mandrel, or alternatively by using several mandrels spaced apart. Any degree of pressure on the structure may be used, and in particular is about 100 psi to about 700 psi, and most preferably of from about 300 psi to about 500 psi. The pressure exerted may be reduced if a less dense structure is desired, or if the mechanical compressor 60 is heated

The structure may have any desired thickness, the thickness being measured from the outer surface of the structure to the inner surface. Preferably, the structures herein have a thickness from about 5 mm to about 30 mm, and most preferably from about 8 mm to about 14 mm.

After compression of the ePTFE structure, the compressed structure may be formed into any shape or prosthetic desired. As with the pre-compressed structure, the compressed structure may be tubular, or it may be sheet-like, or any combination thereof. A tubular compressed structure may be thin or thick, may be circular in diameter or it may be oval or other shape, and it may be tapered or flared, or combinations thereof. It may additionally be multi-structural, such as the bifurcated system for AAA repair, or a multi-lumen structure, both described above.

The structure may incorporate more than one layer of ePTFE, which may be sintered or bonded together if desired. The layers of ePTFE may be sintered or bonded prior to or after mechanical compression. In addition, the ePTFE structure may additionally incorporate additional layers, including a stent layer. The stent layer may be of any stent configuration known to those skilled in the art, including those used alone or in a stent-graft arrangement. Various stent types and stent constructions may be employed in the present invention including, without limitation, self-expanding stents and balloon expandable stents. The stents may be capable of radially contracting as well. Self-expanding stents include those that have a spring-like action which cause the stent to radially expand or stents which expand due to the memory properties of the stent material for a particular configuration at a certain temperature. Other materials are, of course, contemplated, such as stainless steel, platinum, gold, titanium, tantalum, niobium, nitinol and other biocompatible materials, as well as polymeric stents. The configuration of the stent may also be chosen from a host of geometries. For example, wire stents can be fastened in a continuous helical pattern, with or without wave-like forms or zigzags in the wire, to form a radially deformable stent. Individual rings or circular members can be linked together such as by struts, sutures, or interlacing or locking of the rings to form a tubular stent. Furthermore, stents may be formed by etching a pattern into a material or mold and depositing stent material in the pattern, such as by chemical vapor deposition or the like. Examples of various stent configurations are shown in U.S. Pat. No. 4,503,569 to Dotter; U.S. Pat. No. 4,733,665 to Palmaz; U.S. Pat. No. 4,856,561 to Hillstead; U.S. Pat. No. 4,580,568 to Gianturco; U.S. Pat. No. 4,732,152 to Wallsten, U.S. Pat. No. 4,886,062 to Wiktor, and U.S. Pat. No. 5,876,448 to Thompson, all of whose contents are incorporated herein by reference.

The stent layer may be used in conjunction with one or more layers of ePTFE, and may be incorporated prior to or after mechanical compression. In one embodiment, the stent layer is on the inside surface of the tubular ePTFE structure. In another embodiment, the stent layer is on the outside surface of the tubular ePTFE structure. In yet another embodiment, the stent layer is sandwiched between overlapping layers of ePTFE. The stent layers and multiple layers of ePTFE may overlap each other fully or partially, whichever structure is desired. In some embodiments several stent layers may be incorporated.

In another embodiment, there is contemplated multiple, separated stent layers sandwiched between multiple layers of ePTFE, providing a structure that has a section incorporating a stent, as well as concurrent section that is devoid of a stent portion. Other layers of material may optionally be incorporated into the ePTFE structure, including non-porous films, or other, more porous ePTFE layers. In one embodiment, there may be multiple layers of ePTFE, each layer having a varied density.

The layers of ePTFE and/or stent layers are preferably sintered together to add stability to the layered structure. Sintering of the structure may be achieved by any means desired. In a preferred embodiment, the structure is heated at a high temperature, approximately 625° F., for a sufficient time to effectively seal the compressed structure. The sintering is typically conducted for about 10 to about 15 minutes, and more specifically about 12 minutes in total. Once sintered, the structure may be collected for later use. Combinations of heat and pressure may be used to achieve sintering of the layers together. For example, temperatures of from about 600° F. to about 750° F. may be used for sintering.

In addition, other layers may optionally be incorporated to the structure for added stability. In one embodiment, the structure may incorporate a layer of silicone, which may be sintered on the surface of the ePTFE structure. The silicone may be on the inside or the outside of the ePTFE structure. In embodiments incorporating a stent on the outer surface of the ePTFE structure, the silicone layer may overlap the stent layer of the structure. There may optionally be multiple layers of silicone incorporated on or in the ePTFE structure. Further, the silicone layer may fully cover the structure, or it may only partially cover the structure. The silicone layer or layers may be sintered to the ePTFE structure at any location or locations desired. Sintering of the silicone layer may be achieved by any method, including that described above for sintering of the ePTFE structure.

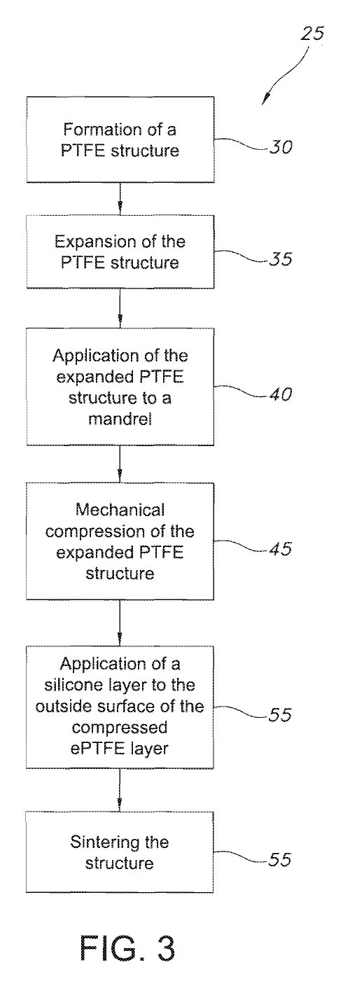

With reference to FIG. 3, a schematic description of one embodiment of the process 25 of forming a densified tube is shown. In a first step 30, a structure of PTFE is formed. Any means to form the PTFE structure may be used, preferably extrusion of PTFE resin is used. Further, the PTFE structure may be of any shape or style contemplated. In the preferred embodiment, the PTFE structure is tubular. In a next step 35, the PTFE structure is expanded to form a structure of ePTFE. Any means of expansion may be used to achieve expansion. In a next step 40, the ePTFE structure is applied to the surface of a mandrel, or other surface which can withstand compressive forces. A next step 45 contemplates mechanical compression of the expanded PTFE structure on the mandrel. In a preferred embodiment, the ePTFE structure is tubular and is applied to the outer surface of a rod-shaped mandrel, where a mechanical compressor as described in more detail above is used to exert pressure to the outside surface of the ePTFE structure. The mandrel may be made of any material desired, and may include holes or dimples on the surface of the mandrel. Holes may be incorporated on the mandrel, to allow for proper lamination, giving acceptable heat transfer and acceptable air release. The mandrel may additionally incorporate dimpled regions surrounding the hole. Dimples on the mandrel may add flexibility to the completed ePTFE structure.

Optionally, a stent may be applied to the ePTFE structure prior to or after the compression step 45. The exerted pressure compresses the ePTFE structure, forcing it to a thinner, denser state. In a next step 50, an optional layer of silicone is applied to the outside of the ePTFE structure. In a final step 55, the layered and compressed ePTFE structure is sintered via application of heat as described above. Sintering holds the densified and layered ePTFE structure in place for later use. Optionally, there may be several layers of ePTFE, and there may be at least one stent layer incorporated into the structure.

While the invention has been described by reference to certain preferred embodiments, it should be understood that numerous changes could be made within the spirit and scope of the inventive concept described. Accordingly, it is intended that the invention not be limited to the disclosed embodiments, but that it have the full scope permitted by the language of the following claims.

What is claimed is:

1. A method for manufacturing compressed ePTFE, comprising the steps of:
a. providing a layer of ePTFE;
b. applying said layer of ePTFE to a mandrel;
c. mechanically compressing said layer of ePTFE on said mandrel; and
d. removing said compressed ePTFE;
wherein said compressed ePTFE is denser than uncompressed ePTFE.

2. The method of claim 1, wherein said compressed ePTFE is tubular in shape.

3. The method of claim 1, further comprising the step of at least partially covering said compressed ePTFE with a layer of silicone.

4. The method of claim 3, further comprising the step of at least partially sintering said compressed ePTFE and said silicone layer.

5. The method of claim 4, wherein said sintering is achieved by heating said ePTFE and said silicone layer at a temperature of about 625° F. for about 10 to about 12 minutes.

6. The method of claim 1, wherein said step of mechanically compressing said layer of ePTFE on said mandrel comprises exerting a pressure on said ePTFE of about 300 to about 500 psi.

7. The method of claim 1, wherein said compressed ePTFE has a WEP value of at least about 20 psi.

8. The method of claim 1, wherein said compressed ePTFE has an average density of from about 1.0 to about 1.5 grams/cc.

9. An ePTFE tube, comprising at least one layer of compressed ePTFE, wherein said ePTFE has a WEP value of at least about 20 psi and a density of from about 1.0 to about 1.5 grams/cc.

10. The tube of claim 9, further comprising a layer of silicone at least partially covering the outside of said tube.

11. The tube of claim 9, further comprising a stent layer.

12. The tube of claim 9, further comprising at least one additional layer of ePTFE, wherein said additional layer of ePTFE comprises a WEP value of at least about 20 psi and a density of from about 1.0 to about 1.5 grams/cc.

13. The tube of claim 12, further comprising a stent between said layers of ePTFE.

14. A prosthetic structure, comprising at least one layer of ePTFE, wherein said ePTFE comprises a WEP value of at least about 20 psi and a density of about 1.0 to about 1.5 grams/cc.

15. The prosthetic structure of claim 14, wherein said prosthetic structure is selected from the group consisting of a tube, sheet, tape, or combinations thereof.

16. The prosthetic structure of claim 15, wherein said tube is round, oval, tapered, flared, or other non-cylindrical shape.

17. The prosthetic structure of claim 14, wherein said prosthetic structure is adapted to repair an abdominal aortic aneurysm.

18. The prosthetic structure of claim 14, wherein said prosthetic structure is adapted to repair a thoracic aortic aneurysm.

19. The prosthetic structure of claim 14, further comprising a stent layer.

20. The prosthetic structure of claim 19, wherein said stent layer is located between two layers of said ePTFE.

21. The prosthetic structure of claim 14, further comprising a layer of silicone at least partially covering said layer of ePTFE.

22. An apparatus for conveying fluid, comprising an ePTFE tube, said ePTFE tube comprising at least one layer of compressed ePTFE, wherein said ePTFE has a WEP value of at least about 20 psi and a density of from about 1.0 to about 1.5 grams/cc.

23. The apparatus of claim 22, further comprising a layer of silicone at least partially covering the outside of said tube.

24. The apparatus of claim 22, further comprising a stent layer.

25. The apparatus of claim 22, further comprising at least one additional layer of ePTFE, wherein said additional layer of ePTFE comprises a WEP value of at least about 20 psi and a density of from about 1.0 to about 1.5 grams/cc.

26. The apparatus of claim 25, further comprising a stent between said layers of ePTFE.

27. A means for conveying fluid, comprising an ePTFE tube, said ePTFE tube comprising at least one layer of compressed ePTFE.
